(19) United States
(12) Patent Application Publication
(10) Pub. No.: US 2025/0009639 A1
(43) Pub. Date: Jan. 9, 2025

(54) ANTIMICROBIAL, ANTIFUNGAL, AND SKIN MOISTURIZING COMPOSITION

(71) Applicant: Nabil Hannoush, Westfield, MA (US)

(72) Inventor: Nabil Hannoush, Westfield, MA (US)

(21) Appl. No.: 18/765,811

(22) Filed: Jul. 8, 2024

Related U.S. Application Data
(60) Provisional application No. 63/512,387, filed on Jul. 7, 2023.

(51) Int. Cl. A61K 8/92; A61K 8/36; A61K 8/42; A61Q 17/00; A61Q 19/00

(52) U.S. Cl. CPC A61K 8/922; A61K 8/36; A61K 8/42; A61Q 17/005; A61Q 19/007; A61K 2800/5922; A61K 2800/87

(57) ABSTRACT

A composition for treating the human skin, for example the skin of the human foot, having a therapeutically effective amount of one or more weak organic acids compatible with human skin delivered at a pH of below 3.5, the weak organic acid includes at least one of acetic acid and vinegar, and the composition includes urea.

### CROSS-REFERENCE TO RELATED APPLICATIONS

This application claims the benefit of U.S. Provisional Application No. 63/512,387 filed Jul. 7, 2023, the disclosure of which is incorporated herein by reference in its entirety.

### BACKGROUND

The disclosed embodiments relate to an antimicrobial and an antifungal composition, and more particularly to an antimicrobial and an antifungal composition that includes urea and has a pH that complements the natural pH of healthy skin, to thereby treat and maintain the health of the skin, and for example, the skin of the foot.

Unlike other areas of the human skin, the skin of the foot may be exposed to relatively low levels of light and to relatively high levels of moisture throughout the day. Such exposure may increase the skin's susceptibility to microbial or fungal infections. Similarly, individuals who wear sandals or other foot exposing shoes, may suffer from skin conditions such as atopic dermatitis which may result from environmental and/or chemical exposures, and which may be exacerbated by microbial agents.

Commercially available products on the market may be used to resolve bacterial or fungal foot infections. However, these products may not simultaneously treat and prevent both bacterial and fungal infections and moisturize the skin as their formulations may not account for the natural pH of healthy skin.

Some home remedies may use food grade vinegar as an antifungal remedy for the foot. However, these remedies may require long exposure times, i.e., bathing the foot in the vinegar solution for approximately 30 minutes at a time over a period of months. Such treatment times may be inconvenient. In addition, such remedies may require the utilization of a heat source to supply heat, which may be inconvenient and costly. Such remedies may cause damage to the stratum corneum which produces natural skin protecting oils, which may cause the skin to become dried and cracked. Such skin conditions may increase the risk of the skin developing microbial infections.

For example, the human epidermis may have a pH of about 4.0 to about 4.3. Conventionally known skin products may have a pH of greater than 7.0. This relatively high pH level may destroys the skin's acid mantle. This may cause the skin's pH to increase, which in turn may causes dryness of the peripheral layers of the skin.

### BRIEF SUMMARY

The disclosed embodiments provide an antifungal, antibacterial, and moisturizing composition, in which urea is an element, and the composition may be useful in the care of the skin, and for example in the care of the skin of the human foot. The composition may have an efficacy against bacteria and fungi, and may be formulated to have acidic properties. In one embodiment, the composition may have a pH of below about 3.5. In one embodiment, the composition may have a pH in the range of about 3.0 to about 3.2. As the composition may have a pH which is compatible with the pH of healthy skin, it may create an essentially homeopathic treatment for a variety of skin conditions caused by bacterial and fungal infections, and for example to those conditions associated with the skin of the foot. The composition may be used in combination with a substrate to achieve an effective skin treatment. An exemplary substrate may include one or more of rayon and polyester fibers. In an exemplary embodiment, the substrate may be packaged as a plurality of individual wipes which may be impregnated with the composition to treat and prevent bacterial and fungal causing skin conditions, such as, e.g., tinea pedis, bromhidrosis, hyperhidrosis, eczema, pompholyx, atopic dermatitis, and the like.

More specially, disclosed is a composition for the prevention and treatment of skin conditions caused by bacteria and fungus, the composition includes: an aqueous solution including vinegar; and urea, wherein the composition has a pH of below 3.5.

In addition to one or more aspects of the composition, or as an alternate, the composition has a pH of 2.9 to 3.2.

In addition to one or more aspects of the composition, or as an alternate, the composition has a pH of 3.1.

In addition to one or more aspects of the composition, or as an alternate, the composition further includes 0.04-0.08 weight percent of citric acid, wherein the weight percent is based upon the total weight of the composition.

In addition to one or more aspects of the composition, or as an alternate, the composition further includes an essential oil, wherein the essential oil includes one or more of argan oil, lavender oil, rose oil, tea tree oil, jojoba oil, and carrot seed oil.

In addition to one or more aspects of the composition, or as an alternate, the composition further includes an extract, wherein the extract includes one or more of lavender extract and rose extract.

In addition to one or more aspects of the composition, or as an alternate, the composition includes 10 weight percent of vinegar, wherein the weight percent of the vinegar is based upon the total weight of the composition.

In addition to one or more aspects of the composition, or as an alternate, the composition further includes 1.0-3.0 weight percent of glycerin, wherein the weight percent of the glycerin is based upon the total weight of the composition.

In addition to one or more aspects of the composition, or as an alternate, the composition further includes 0.12 to 0.24 weight percent of an essential oil, wherein the weight percent of the essential oil is based upon the total weight of the composition.

In addition to one or more aspects of the composition, or as an alternate, the composition further includes 0.10-0.20 weight percent of an extract, wherein the weight percent of the extract is based upon the total weight of the composition.

In addition to one or more aspects of the composition, or as an alternate, the composition includes: 0.05-0.10 weight percent of lavender oil; 0.05-0.10 weight percent of rose oil; 0.01-0.02 weight percent of tea tree oil; 0.01-0.02 weight percent of carrot seed oil; 0.05-0.01 weight percent of lavender extract; and 0.05-0.01 weight percent of rose extract; and 0.01-0.02 weight percent of urea, wherein the weight percents are based upon the total weight of the composition.

Further disclosed is a wipe for treating and preventing skin conditions caused by bacteria and fungus, wherein the wipe includes: a substrate; and a composition of having one or more of the above disclose aspects, wherein the substrate is saturated with the composition.

In addition to one or more aspects of the wipe, or as an alternate, the pH of the composition is 2.9-3.2.

In addition to one or more aspects of the wipe, or as an alternate, the wipe is saturated with the composition at a 3% add on level.

In addition to one or more aspects of the wipe, or as an alternate, the wipe includes one or more of polypropylene, polyester, and rayon.

In addition to one or more aspects of the wipe, or as an alternate, the composition includes 10 weight percent of vinegar, wherein the weight percent of the vinegar is based upon the total weight of the composition.

In addition to one or more aspects of the wipe, or as an alternate, the composition further includes 1.0-3.0 weight percent of glycerin, wherein the weight percent is based upon the total weight of the composition.

In addition to one or more aspects of the wipe, or as an alternate, the composition further includes 0.12 to 0.24 weight percent of an essential oil, wherein the weight percent of the essential oil is based upon the total weight of the composition.

In addition to one or more aspects of the wipe, or as an alternate, the composition further includes 0.10-0.20 weight percent of an extract, wherein the weight percent of the extract is based upon the total weight of the composition.

In addition to one or more aspects of the wipe, or as an alternate, the composition further includes 0.04-0.08 weight percent of citric acid, wherein the weight percent is based upon the total weight of the composition.

### DETAILED DESCRIPTION

The disclosed embodiments provide composition that has a pH in the range of about 3.0 to about 3.2. In one embodiment, the composition has a pH of about 2.9 to about 3.2. In one embodiment, the composition has a pH of about 3.0 to about 3.1. Such a pH range may be compatible with the pH of the skin in that may maintain the skin in a moisturized and healthy state while serving as an antimicrobial and antifungal agent.

The composition may include a solution of acetic acid. The solution of acetic acid may be formed in water, and for example, in deionized water, or an aqueous solution of vinegar, which may be a food-grade vinegar. In an exemplary embodiment, the composition includes about 10 weight percent of vinegar. Unless otherwise indicated, reference to a weight percent herein is relative to a total weight of the composition.

In an exemplary embodiment, the composition may further include citric acid which may be used to calibrate the pH of the composition. In an exemplary embodiment, the composition may have a pH in the range of about 3.0-3.2. In one embodiment, the composition may have a pH that is about 3.1. In one embodiment, the composition may include about 0.04-0.08 weight percent of citric acid.

The composition may further include glycerin which may aid in water retention and which may provide a moisturizing effect at relatively low pH ranges. In an exemplary embodiment, the composition may include about 1.0-3.0 weight percent of glycerin.

In an exemplary embodiment, the composition may also include one or more of an essential oil and an extract. Exemplary essential oils may include one or more of argan oil, lavender oil, rose oil, tea tree oil, jojoba oil, and carrot seed oil, and an exemplary extract may include one or more of lavender extract and rose extract. In one embodiment, the composition may include about 0.12-0.24 weight percent of essential oil, and about 0.10-0.20 weight percent of an extract.

Argan oil (Argania Spinosa kernel oil) may be utilized for its skin antioxidant and moisturizing properties. Rose oil and rose extract may be utilized for their antibacterial properties. Lavender oil and lavender extracts may be utilized as mild antiseptics and anti-inflammatory agents. Tea tree oil (Melaleuca alternifolia), distilled from the leaves of the Australian tree, may be utilized as a natural fungicide because of its antiseptic and germicidal properties. Carrot seed oil may be utilized as an antioxidant. Jojoba oil is a plant derived moisturizer and may be utilized as an anti-inflammatory agent.

The composition may also include agents such as at least one or more of an acidifier, a preservative, an additive, and the like. An exemplary acidifier may include, e.g., gloconolactone and an exemplary preservative may include, e.g., sodium benzoate. In an exemplary embodiment, the composition may include about 0.7-1.5 weight percent of such one or more agents.

The composition may also include urea. In an exemplary embodiment, the composition may include about 0.01-0.02 weight percent of urea. This added ingredient may provide moisturization, enhancing the functionality of the active ingredients.

In an exemplary embodiment, the composition includes the components and the amounts set forth in Table 1.

TABLE 1

| Components | Weight Percent |
|---|---|
| Deionized Water | 85.00-88.00 |
| Vinegar | 10.00 |
| Glycerin | 1.0-3.0 |
| Gluconolactone | 0.5-1.0 |
| Sodium Benzoate | 0.20-0.50 |
| Lavender Oil | 0.05-0.10 |
| Rose Oil | 0.05-0.10 |
| Rose Extract | 0.05-0.10 |
| Lavender Extract | 0.05-0.10 |
| Citric Acid | 0.04-0.08 |
| Tea Tree Oil | 0.01-0.02 |
| Carrot Seed Oil | 0.01-0.02 |
| Urea | 0.01-0.02 |

The use of vinegar in the composition may be beneficial as an antifungal and an antibacterial agent as compared to compositions that contain citric acid as a primary component. As indicated, urea provides moisturization, enhances the functionality of the active ingredients. Additionally, the pH level of the composition may create the proper balance between the composition's antifungal, antibacterial, and moisturizing properties.

The disclosed composition may have several benefit. For example, when the pH of the composition is between 3.0-3.1, antifungal results may be found in a relatively short treatment time that has a minimal effect on the stratum corneum. This may reduce the risk of contracting a bacterial infection.

Such antifungal results may be particularly beneficial to persons suffering from bromhidrosis which is caused by the binding of sweat gland secretions with the biofilm of certain fungi. Use of the composition may disrupt the biofilm of the fungi. This may diminish or eliminate the condition. The composition may further treat skin conditions caused by fungi dermatophytes such as Trichophyton, Microsporum and Epirdermophyton, as well as Malassezia and ringworm.

Additionally, the composition may have beneficial results in treating bacterial infections that cause thickened skin, itching, and dryness, and those bacterial infections that may be caused by Staphylococcus aureus which include, e.g., eczema, pompholyx, atopic dermatitis, and the like. Treatment with the disclosed composition may result in skin softening in individuals having these conditions.

The composition may also prevent the occurrence fungal and bacterial infections. Such prevention may be beneficial to individuals who are immune-compromised and/or have poor nutrient diets, as the skin of such individuals may be alkaline. Use of the composition may provide the proper pH to the skin and, therefore, assists in restoring the skin to its healthy, natural PH levels.

According to yet another embodiment, the composition may be incorporated into a substrate such as a wipe, a bandage, gauze, clothing, a drape, bedding, a dressing, a film, a pad or a patch. Where a wipe is used, an exemplary material for the wipe may include synthetic fiber in woven, non-woven or knitted fibers, or blends thereof. Fibers may include one or more of polypropylene, polyester, and rayon. The substrate may be sold under the trademark Geogard, owned by Arxada, LLC, of Morristown New Jersey USA.

In an exemplary embodiment, the wipe may exfoliate the skin and, therefore, remove the macro molecular environmental debris and dead skin cells from the body, along with the superficial fungus and bacterial debris of the epidermis. Once this bacterial- and fungal-protective covering is removed, the composition can then more effectively kill the fungus and bacteria located in the dermis of the skin.

In an exemplary embodiment, the substrate may be impregnated with the disclosed composition. In one embodiment, the composition may be impregnated through a wetting bar system that distributes an equal amount of the composition onto each sheet of substrate. The wipes may be saturated at about a 3% add on level, i.e., at about 3 times the weight of a dry stack of the substrate.

In an exemplary embodiment, the wipes may be packaged in a packaging that may allow the wipes to be manually dispensed from the packaging. Due to the pH of the composition, the impregnated substrate may be packaged in a container with an air-lock plastic seal, and in a high grade polypropylene container. This may resist deterioration.

An exemplary method of using the wipe for preventative purposes may include wiping the outer skin of the foot with a single wipe for approximately 20 seconds per foot taking care to apply the wipe between and around the toes and over the ventral and dorsal sides of the foot. This cleaning may be performed once a day over consecutive days.

Although the composition and the saturated substrates disclosed herein have been described as being particularly beneficial in the application to the human foot, its application may also be beneficial to the application to the skin, especially the paws and hoofs, of a wide range of animals whose skin pH is below about 7.5, including for example, dogs, cats, horses, and the like.

Thus, the embodiments provide a composition for caring for the skin, and particularly for the skin of the human foot. Such composition can be applied in a relatively simple and efficient manner.

In describing exemplary embodiments, specific terminology is employed for the sake of clarity. The embodiments, however, are not intended to be limited to the specific terminology so selected, and it is to be understood that each specific element includes all technical equivalents that operate in a similar manner to accomplish a similar purpose.

The forms of the composition according to the disclosed embodiments are not restricted to the specific forms, and accordingly they may include, for example, a liquid form, an emulsified cream form, a gel form, a solid form, and the like.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

Those of skill in the art will appreciate that various example embodiments are shown and described herein, each having certain features in the particular embodiments, but the present disclosure is not thus limited. Rather, the present disclosure can be modified to incorporate any number of variations, alterations, substitutions, combinations, sub-combinations, or equivalent arrangements not heretofore described, but which are commensurate with the scope of the present disclosure. Additionally, while various embodiments of the present disclosure have been described, it is to be understood that aspects of the present disclosure may include only some of the described embodiments. Accordingly, the present disclosure is not to be seen as limited by the foregoing description, but is only limited by the scope of the appended claims.

What is claimed is:

1. A composition for the prevention and treatment of skin conditions caused by bacteria and fungus, the composition comprising:
an aqueous solution comprising vinegar; and
urea,
wherein the composition has a pH of below 3.5.

2. The composition of claim 1, wherein the composition has a pH of 2.9 to 3.2.

3. The composition of claim 2, wherein the composition has a pH of 3.1.

4. The composition of claim 3, wherein the composition further comprises 0.04-0.08 weight percent of citric acid, wherein the weight percent is based upon the total weight of the composition.

5. The composition of claim 4, wherein the composition further comprises an essential oil, wherein the essential oil comprises one or more of argan oil, lavender oil, rose oil, tea tree oil, jojoba oil, and carrot seed oil.

6. The composition of claim 1, wherein the composition further comprises an extract, wherein the extract comprises one or more of lavender extract and rose extract.

7. The composition of claim 1, wherein the composition comprises 10 weight percent of vinegar, wherein the weight percent of the vinegar is based upon the total weight of the composition.

8. The composition of claim 7, wherein the composition further comprises 1.0-3.0 weight percent of glycerin, wherein the weight percent of the glycerin is based upon the total weight of the composition.

9. The composition of claim 8, wherein the composition further comprises 0.12 to 0.24 weight percent of an essential oil, wherein the weight percent of the essential oil is based upon the total weight of the composition.

10. The composition of claim 9, wherein the composition further comprises 0.10-0.20 weight percent of an extract, wherein the weight percent of the extract is based upon the total weight of the composition.

11. The composition of claim 10, wherein the composition comprises:
0.05-0.10 weight percent of lavender oil;
0.05-0.10 weight percent of rose oil;
0.01-0.02 weight percent of tea tree oil;
0.01-0.02 weight percent of carrot seed oil;
0.05-0.01 weight percent of lavender extract; and
0.05-0.01 weight percent of rose extract; and
0.01-0.02 weight percent of urea,
wherein the weight percents are based upon the total weight of the composition.

12. A wipe for treating and preventing skin conditions caused by bacteria and fungus, wherein the wipe comprises:
a substrate; and
the composition of claim 1,
wherein the substrate is saturated with the solution.

13. The wipe of claim 12, wherein the pH of the composition is 2.9-3.2.

14. The wipe of claim 13, wherein the wipe is saturated with the composition at a 3% add on level.

15. The wipe of claim 14, wherein the wipe comprises one or more of polypropylene, polyester, and rayon.

16. The wipe of claim 12, wherein the composition comprises 10 weight percent of vinegar, wherein the weight percent of the vinegar is based upon the total weight of the composition.

17. The wipe of claim 16, wherein the composition further comprises 1.0-3.0 weight percent of glycerin, wherein the weight percent is based upon the total weight of the composition.

18. The wipe of claim 17, wherein the composition further comprises 0.12 to 0.24 weight percent of an essential oil, wherein the weight percent of the essential oil is based upon the total weight of the composition.

19. The wipe of claim 18, wherein the composition further comprises 0.10-0.20 weight percent of an extract, wherein the weight percent of the extract is based upon the total weight of the composition.

20. The wipe of claim 19, wherein the composition further comprises 0.04-0.08 weight percent of citric acid, wherein the weight percent is based upon the total weight of the composition.
